(19) United States
(12) Patent Application Publication
(10) Pub. No.: US 2011/0152648 A1
(43) Pub. Date: Jun. 23, 2011

(54) PULSE OXIMETER SENSOR

(76) Inventor: Joseph M. Rustick, Phoenix, AZ (US)

(21) Appl. No.: 12/643,659

(22) Filed: Dec. 21, 2009

(51) Int. Cl. A61B 5/1455

(52) U.S. Cl. 600/322

(57) ABSTRACT

A method of sensing or measuring various characteristics of blood is disclosed. The method includes providing a sensor having an emitter and a receiver coupled to external processing apparatus. The sensor has a generally elongated configuration designed to be formed into a generally U-shaped orientation so that the sensor can be positioned over the skin of a patient adjacent the patient's mouth with the emitter and receiver in an aligned orientation on opposite sides of the skin. One of the emitter and receiver is positioned against the skin outside of the mouth and the other is positioned against the skin on the inside of the mouth. The sensor is used to sense or measure at least one selected characteristic of the blood of the patient.

### Field Of The Invention

This invention relates to sensors for sensing the condition or various characteristics of blood, including flow, content, etc.

### BACKGROUND OF THE INVENTION

In the medical industry there are many sensors for measuring blood flow, blood content, heart rate, etc. In most of these, a sensor is temporarily affixed to the end of a finger so as to sense the blood flowing in the finger. The information or data sensed is transmitted through connecting circuitry to processing apparatus that uses the data to produce useful or desired information. Generally, the sensor includes some type of transmitter or emitter that is situated on one side of the finger and a receiver or receptor situated on an opposite side of the finger to receive the emissions.

A major problem with this type of sensor in emergency rooms, on the operating table, etc. is that the flow of blood in the finger may be substantially reduced or even nonexistent. For example, in cases of hypothermia or hypovolemia, the flow of blood in the distal extremities is virtually nonexistent. Thus, in such cases the use of the finger sensors is not possible. Also, in many situations a patient's fingers may be inaccessible (e.g. lost limbs, burn patients, etc.) or impractical in the field of surgery for some reason.

It would be highly advantageous, therefore, to remedy the foregoing and other deficiencies inherent in the prior art.

Accordingly, it is an object of the present invention to provide a new and improved blood sensor.

Another object of the invention is to provide a new and improved sensor that can be used on patients in all circumstances and especially in cases of hypovolemia and hypothermia.

Another object of the invention is to provide a new and improved sensor that is convenient to use and can be quickly and easily engaged on a patient.

### SUMMARY OF THE INVENTION

Briefly, to achieve the desired objects of the instant invention in accordance with a preferred embodiment thereof a method of sensing or measuring various characteristics of blood is disclosed. The method includes providing a pulse oximeter or other sensor depending on blood flow and having an emitter and a receiver coupled to external processing apparatus. The sensor has a generally elongated configuration designed to be formed into a generally U-shaped orientation so that the sensor can be positioned over the skin of a patient adjacent the patient's mouth with the emitter and receiver in an aligned orientation on opposite sides of the skin. One of the emitter and receiver is positioned against the skin outside of the mouth and the other is positioned against the skin on the inside of the mouth. The sensor is used to sense or measure at least one selected characteristic of the blood of the patient.

The desired objects of the instant invention are further achieved with a pulse oximeter sensor that includes a generally elongated thin body designed to be formed into a generally U-shaped orientation and having a thin formable base layer. An emitter is positioned on the base layer adjacent one end thereof and further positioned to emit outwardly generally away from the base layer. A receiver is positioned on the base layer adjacent an opposite end thereof and further positioned to receive emissions directed generally at the base layer. The elongated thin body is designed to be positioned over the skin of a patient adjacent the patient's mouth in the generally U-shaped orientation, the emitter and the receiver being further positioned to be aligned in direct communication through the patient's skin with one of the emitter and receiver positioned outside the patient's mouth and the other of the receiver and the emitter inside the patient's mouth.

### BRIEF DESCRIPTION OF THE DRAWING

The foregoing and further and more specific objects and advantages of the instant invention will become readily apparent to those skilled in the art from the following detailed description of a preferred embodiment thereof taken in conjunction with the drawings, in which:

FIG. 1 is a perspective view of a pulse oximeter sensor in accordance with the present invention;

FIG. 2 is a side view of the pulse oximeter sensor of FIG. 1;

FIG. 3 is a sectional view of the pulse oximeter sensor of FIG. 1, as seen from the line 3-3 in FIG. 1;

FIG. 4 is a sectional view of the pulse oximeter sensor of FIG. 1, as seen from the line 4-4 in FIG. 1;

FIG. 5 is a perspective view of the pulse oximeter sensor of FIG. 1 in an operating orientation;

FIG. 6 is a sectional view of the pulse oximeter sensor of FIG. 5 in the operating orientation;

FIG. 7 is a perspective view of the pulse oximeter sensor of FIG. 5 as it appears in the operating orientation on a patient; and

FIG. 8 is an enlarged sectional view of the pulse oximeter sensor of FIG. 5 in the operating orientation.

### DETAILED DESCRIPTION OF A PREFERRED EMBODIMENT

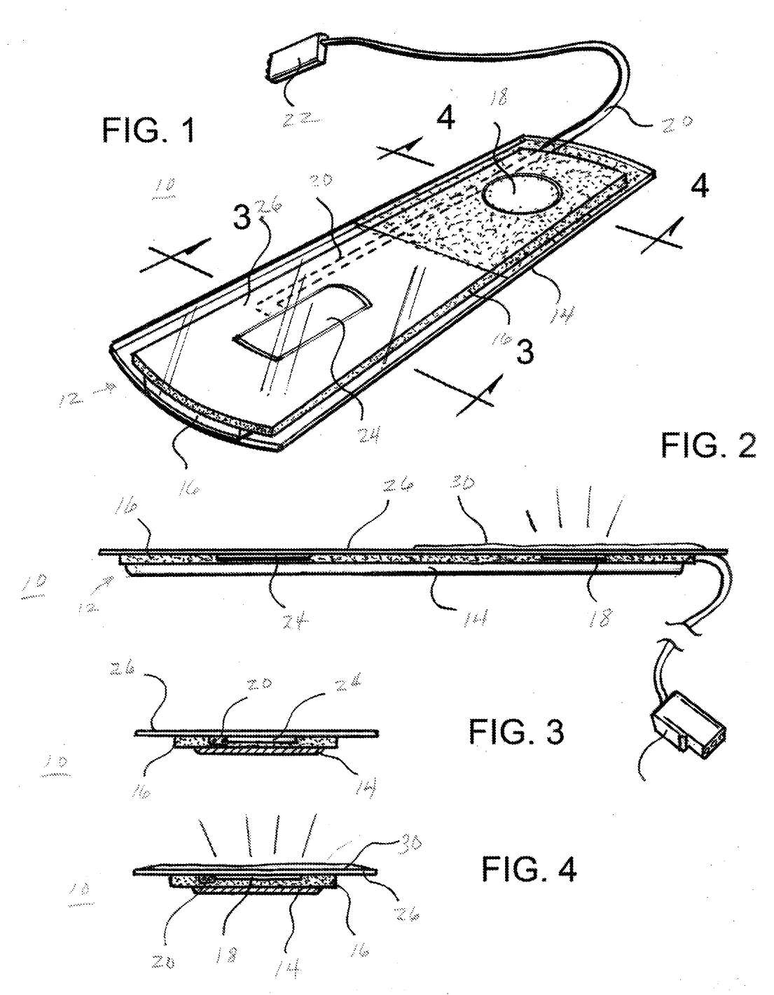

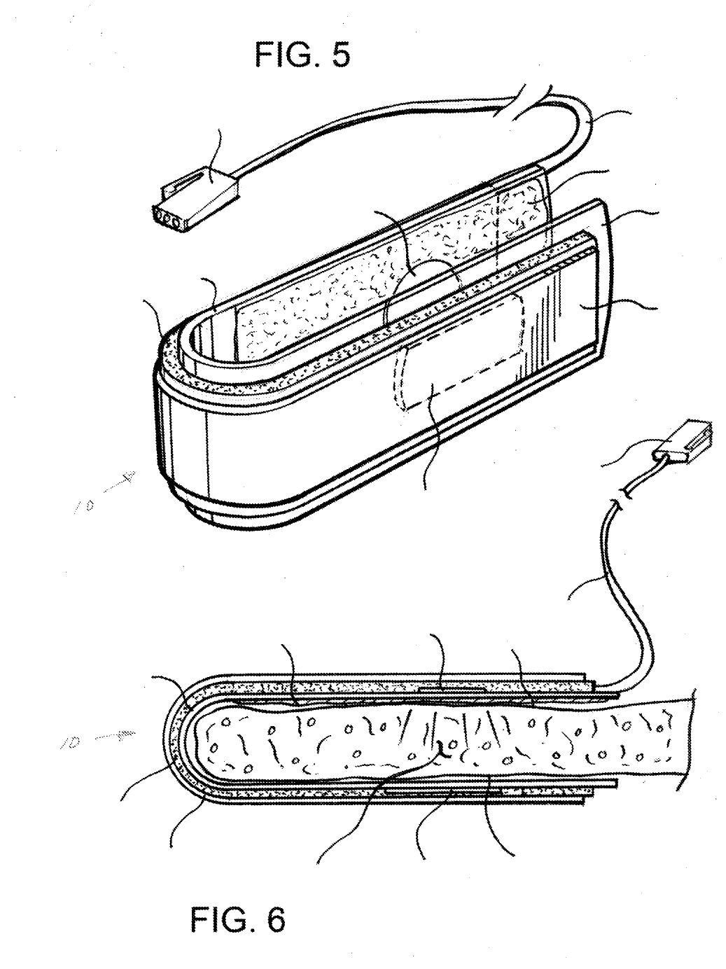

Turning now to FIGS. 1-4, several views are provided of one embodiment of a pulse oximeter sensor 10, in accordance with the present invention. It will be understood that while a pulse oximeter sensor is the preferred sensor in the present invention, other sensors which depend on blood flow can be employed. In this preferred embodiment, sensor 10 includes an elongated generally rectangularly shaped body 12 having a thin formable base layer 14 on the outer side. Formable base layer 14 can be any convenient material provided to hold sensor 10 in a desired orientation and may include, for example, metal, hard or firm plastic, etc. that can be bent into a tight U-shape (e.g. see FIG. 5) and will operate substantially as a spring-clip. In an alternative embodiment, formable base layer 14 can simply be a relatively rigid substrate on which the remaining components are mounted.

A transmitter or emitter 18 is affixed to the inner surface of formable base layer 14, adjacent the right hand end in FIGS. 1 and 2, so as to direct emissions outwardly away from the upper or inner surface of formable base layer 14. It will be understood by those skilled in the art that emitter 18 can be a light source (e.g. visible, infrared, etc.), an ultra-sonic source, a microwave source, etc. Also, emitter 18 is electrically connected through a hard wired coupling 20 and a connector 22 to external processing apparatus (not shown) that pulses emitter 18 to produce digital data.

A receiver 24 is also affixed to the inner surface of formable base layer 14, adjacent the left hand end in FIGS. 1 and 2, so as to receive emissions from emitter 18, as will be explained in more detail presently. It will be understood by those skilled in the art that receiver 24 is designed to operate in conjunction with emitter 18 and will be compatible therewith. Also, receiver 24 is electrically connected through a hard wired coupling 20 and a connector 22 to external processing apparatus (not shown) that activates receiver 24 or otherwise prepares receiver 24 for operation.

A layer 16 of pliable padding material is positioned on the inner or upper surface of formable base layer 14 surrounding emitter 18 and receiver 24 and provides a conformable surface for engagement of sensor 10 with a patient. Layer 16 could be or could include, for example, a soft or foam rubber, plastic, cloth, etc. Basically, layer 16 allows sensor 10 to conform to the portion of the patient's body to which sensor 10 is affixed. A very thin, layer 26 of smooth material is fixed to the upper or inner surface of layer 16 to allow sensor 10 to be easily cleaned and used multiple times. While layer 26 is illustrated in this example as simply covering the upper or inner surface of sensor 10, it will be understood that layer 26 could completely surround sensor 10 on all surfaces to make sensor 10 more susceptible to cleaning and/or sterilizing for future uses. It should be noted that the entire structure of sensor 10, i.e. layers 14, 16, and 26, are preferably less than approximately three eighths (⅜) of an inch.

In at least some applications it may be expedient to apply a thin layer 30 of conductive material over a portion of the emitter end, including emitter 18, designed and included to improve conduction from emitter 18 to the skin of a patient. Materials of this type are well known in the art and may, for example, include some form of petroleum gel or organic material.

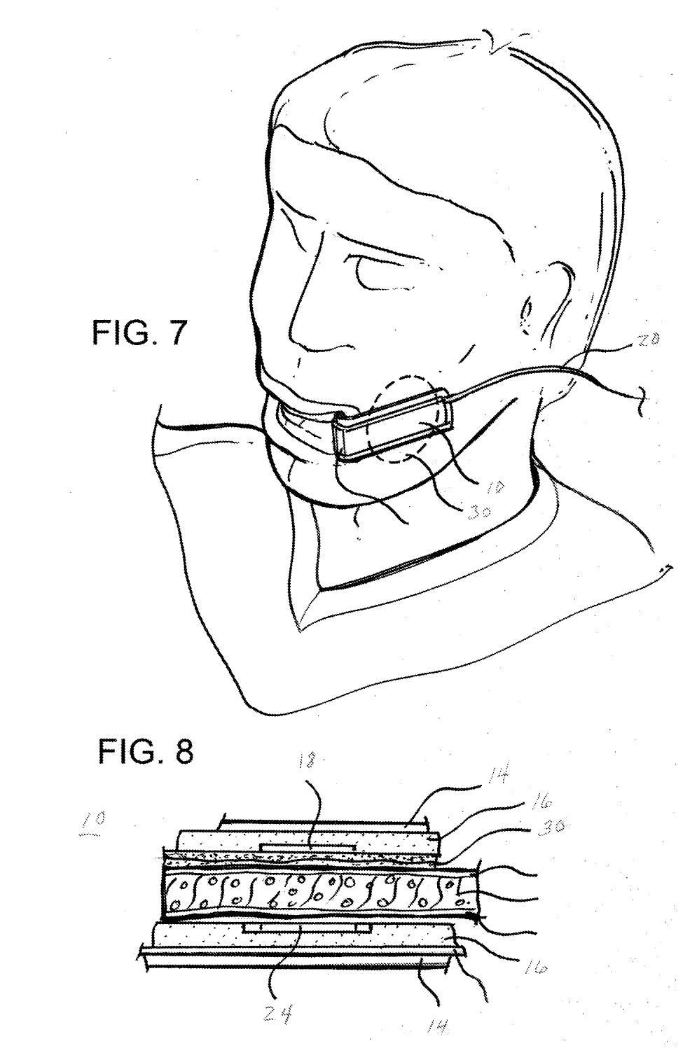

In operation, pulse oximeter sensor 10 is folded to form a generally U-shaped configuration as illustrated in FIG. 5. Sensor 10 is then positioned over a lip or cheek as illustrated in FIG. 7. In a preferred embodiment, formable base layer 14 is constructed to operate like a spring clip and gently holds sensor 10 firmly in engagement with the selected portion of the lip or cheek. In this example the cheek was selected for convenience in illustration. While a spring clip configuration is illustrated here for simplicity and convenience of operation, it will be understood that sensor 10 could be held in place by any other convenient holding structure or apparatus including tape or the like.

Sensor 10 is further positioned with emitter 18 and layer 30 on the outside of the cheek with receiver 24 positioned inside the patient's mouth and directly opposite emitter 18 in an aligned orientation. Thus, if a layer 30 of conductive material is used to enhance conduction from emitter 18 to the cheek skin of the patient, it will be on the outside of the mouth and not create any offensive taste to the patient. In this position, sensor 10 is comfortable for the patient and does not unduly restrict conversation or otherwise cause discomfort to the patient.

Sensor 10 is attached by means of coupling 20 and connector 22 to external processing apparatus (not shown) that pulses emitter 18 to produce digital data, activates receiver 24 or otherwise prepares receiver 24 for operation. Coupling 20 and connector 22 connect data from receiver 24 to the processing apparatus which uses the data to produce useful or desired information about the blood or the desired characteristic of the blood.

Thus, a new and improved pulse oximeter sensor has been disclosed that can be used on patients in all circumstances. Also, the new and improved pulse oximeter sensor is convenient to use and can be quickly and easily engaged on a patient. The pulse oximeter sensor is constructed for maximum efficiency while causing the least discomfort to a patient and is further constructed for easy cleaning and reuse.

Various changes and modifications to the embodiment herein chosen for purposes of illustration will readily occur to those skilled in the art. To the extent that such modifications and variations do not depart from the spirit of the invention, they are intended to be included within the scope thereof which is assessed only by a fair interpretation of the following claims.

Having fully described the invention in such clear and concise terms as to enable those skilled in the art to understand and practice the same, the invention claimed is:

1. A method of sensing or measuring various characteristics of blood including the steps of:
providing a sensor including an emitter and a receiver coupled to external processing apparatus, the sensor having a generally elongated configuration designed to be formed into a generally U-shaped orientation;
positioning the sensor over the skin of a patient adjacent the patient's mouth with the emitter and receiver in an aligned orientation on opposite sides of the skin, one of the emitter and receiver against the skin outside of the mouth and the other of the receiver and the emitter against the skin on the inside of the mouth; and
using the sensor to sense or measure at least one selected characteristic of the blood of the patient.

2. A method as claimed in claim 1 wherein the step of providing a sensor includes providing a sensor with a generally elongated thin body designed to be formed into the generally U-shaped orientation.

3. A method as claimed in claim 2 wherein the step of providing the sensor with the generally elongated thin body designed to be formed into the generally U-shaped orientation includes providing a body including a thin formable base layer, a pliable padding material positioned on a surface of the formable base layer, and a smooth layer of material fixed to a surface of the pliable padding material.

4. A method as claimed in claim 1 wherein the step of providing a sensor includes providing a sensor constructed to form a spring clip and the step of positioning the sensor over the skin of the patient adjacent the patient's mouth includes grasping the skin of the patient adjacent the patient's mouth with the spring clip.

5. A method as claimed in claim 4 wherein the step of providing the sensor constructed to form a spring clip grasping the skin of a patient adjacent the patient's mouth and positioning the emitter and receiver aligned on opposite sides of the skin includes positioning the emitter against the skin outside of the mouth and the receiver against the skin on the inside of the mouth.

6. A method as claimed in claim 5 wherein the step of positioning the emitter and receiver in an aligned orientation on opposite sides of the skin includes applying a conductive material to the smooth layer of material overlying the emitter.

7. A method as claimed in claim 1 wherein the step of positioning the sensor over the skin of the patient adjacent the patient's mouth includes taping the sensor to the skin of the patient adjacent the patient's mouth.

8. A method of sensing or measuring various characteristics of blood including the steps of:
providing a sensor including an emitter and a receiver coupled to external processing apparatus, providing the sensor with a generally elongated thin body including a thin formable base layer, a pliable padding material positioned on a surface of the formable base layer, and a smooth layer of material fixed to a surface of the pliable padding material, the body being designed to be formed into a generally U-shaped orientation;
positioning the sensor over the skin of a patient adjacent the patient's mouth with the emitter and receiver in an aligned orientation on opposite sides of the skin, one of the emitter and receiver against the skin outside of the mouth and the other of the receiver and the emitter against the skin on the inside of the mouth; and
using the sensor to sense or measure at least one selected characteristic of the blood of the patient.

9. A blood sensor comprising:
a generally elongated thin body designed to be formed into a generally U-shaped orientation, the body including a thin formable base layer;
an emitter positioned on the base layer adjacent one end thereof and further positioned to emit outwardly generally away from the base layer;
a receiver positioned on the base layer adjacent an opposite end thereof and further positioned to receive emissions directed generally at the base layer; and
the elongated thin body designed to be positioned over the skin of a patient adjacent the patient's mouth in the generally U-shaped orientation, the emitter and the receiver being further positioned to be aligned in direct communication through the patient's skin with one of the emitter and receiver positioned outside the patient's mouth and the other of the receiver and the emitter inside the patient's mouth.

10. A blood sensor as claimed in claim 9 wherein the emitter and the receiver are connected to external circuitry for receiving actuation signals and for supplying information relative to at least one selected characteristic of blood of a patient.

11. A blood sensor as claimed in claim 9 wherein the body is constructed to form a spring clip designed to fit over the skin of a patient adjacent the patient's mouth and grasp the skin of the patient adjacent the patient's mouth with the spring clip.
